(19) United States
(12) Patent Application Publication
(10) Pub. No.: US 2023/0398171 A1
(43) Pub. Date: Dec. 14, 2023

(54) Natural Composition and Method for Relief from Chronic Arthritis Pain and Inflammation

(71) Applicant: Esperanza Gallego, Sahuarita, AZ (US)

(72) Inventor: Esperanza Gallego, Sahuarita, AZ (US)

(21) Appl. No.: 17/867,753

(22) Filed: Jul. 19, 2022

Related U.S. Application Data
(60) Provisional application No. 63/351,963, filed on Jun. 14, 2022.

(51) Int. Cl. A61K 36/9066; A61K 36/738; A61K 9/00

(52) U.S. Cl. CPC A61K 36/9066; A61K 36/738; A61K 9/0095

(57) ABSTRACT

The present invention relates to natural compositions and methods for providing immediate and effective treatment of chronic pain and inflammation caused due to arthritis. The composition includes a mixture of organic apple juice and turmeric powder in the form of tonics and juices that are consumed by a person affected from arthritis. In some embodiments, the composition is in the form of a powder formed by mixing dry apple powder and turmeric powder. In other embodiments, the composition can be in the form of applesauce, capsules, and more. The composition is fast-acting and provides relief within 15 minutes of consumption. The composition reduces dependency on chemical medications and physical therapies and provides an improved quality of life.

### CROSS-REFERENCE TO RELATED APPLICATION

The present application claims priority to, and the benefit of, U.S. Provisional Application No. 63/351,963, which was filed on Jun. 14, 2022 and is incorporated herein by reference in its entirety.

### FIELD OF THE INVENTION

The present invention relates generally to the field of arthritis and treatment of arthritis pain and inflammation. More specifically, the present invention relates to natural composition for immediate relief from chronic arthritis pain and inflammation. The composition reduces dependency on chemicals, therapies, and surgical procedures. More specifically, the composition has apple and turmeric powder and can be available in form of juice, capsules, powder, applesauce, and more. The composition can be consumed multiple times a day without any side effects. Accordingly, the present disclosure makes specific reference thereto. Nonetheless, it is to be appreciated that aspects of the present invention are also equally applicable to other like applications, devices, and methods of manufacture.

### BACKGROUND

By way of background, arthritis is the swelling and tenderness of one or more joints. The main symptoms of arthritis are joint pain and stiffness, which typically worsen with age. The most common types of arthritis are osteoarthritis and rheumatoid arthritis. In fact, Arthritis isn't a single disease; the term refers to joint pain or joint disease, and there are more than 100 types of arthritis and related conditions. Osteoarthritis is often related to aging or to an injury. Rheumatoid arthritis, or RA, is an autoimmune and inflammatory disease, in which the immune system attacks healthy cells, causing inflammation. RA mainly attacks the joints, usually many joints at once.

Due to arthritis, people face continuous pain and inflammation and avoid activities like running, bicycling, jumping, aerobics, and playing games like tennis and basketball. Medication, physiotherapy, and sometimes surgery help reduce symptoms of arthritis and improve quality of life. Commonly used arthritis medications include nonsteroidal anti-inflammatory drugs (NSAIDs), disease-modifying antirheumatic drugs (DMARDs), counterirritants, steroids and more. Commonly used therapies are cognitive behavioral therapy, relaxation therapy, acupuncture, heat and cold massage, and more.

As a result of use of medication and therapies; tiredness, inflammation, and dehydration can occur. Further, such medications and therapies are expensive and time consuming and may have negative effects on overall health of an individual.

Therefore, there exists a long-felt need in the art for an improved treatment for arthritis that is organic and chemical-free. There is also a long-felt need in the art for an improved treatment for treating arthritis that is safe and cost-effective. Additionally, there is a long-felt need in the art for an arthritis treatment that reduces dependency on conventional arthritis medications and therapies. Moreover, there is a long-felt need in the art for an organic arthritis treatment that is capable of alleviating aches, pains, and stiffness in the joints to promote better mobility. Further, there is a long-felt need in the art for an arthritis treatment that does not require a person to repeatedly visit a medical practitioner. Further, there is a long-felt need in the art for an arthritis treatment that provides immediate relief and helps in improving quality of life of people affected from arthritis. Finally, there is a long-felt need in the art for an arthritis treatment that can be consumed like a tonic and utilizes organic ingredients to improve overall health without the need for chemical medications.

The subject matter disclosed and claimed herein, in one embodiment thereof, comprises a natural composition for relief from chronic arthritis pain. The composition does not contain any chemicals and further comprises a mixture of organic apple juice and turmeric powder, wherein 4-oz of apple juice and 2 grams of turmeric powder are mixed to form a tonic to be consumed for immediate relief from chronic arthritis pain and inflammation. In some embodiments, the composition can be available in form of powder in which dry apple powder and turmeric powder are mixed. The intake of the composition can be increased or decreased depending on the severity of arthritis and provides relief within fifteen minutes of consumption of the composition.

In this manner, the arthritis pain relieving composition of the present invention accomplishes all of the foregoing objectives and provides users with an organic composition capable of alleviating aches, pains, and stiffness in the joints. The composition is natural and helps in improving overall health without the need for chemical medications. The composition is available in different forms and improves quality of life of people suffering from various forms of arthritis.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a composition for relief from chronic arthritis pain and further comprising a mixture of organic apple juice and turmeric powder, wherein 4-oz of apple juice and 2 grams of turmeric powder are mixed to form a tonic to be consumed for immediate relief from chronic arthritis pain and inflammation.

In yet another embodiment, the turmeric powder is extracted by oiling the rhizomes, drying in a hot oven, and then grinding the dried roots.

In yet another embodiment, dry apple powder is mixed with turmeric powder to form the composition in a powder form.

In yet another embodiment, the turmeric powder is mixed in an applesauce wherein applesauce includes natural apple crush.

In yet another embodiment, a chemical-free and natural arthritis antidote composition is disclosed. The antidote is in the form of one of a tonic, juice, applesauce, capsule, or powder and comprising 100% organic apple, pure to 95% turmeric powder, wherein the apple and turmeric powder are mixed in the ratio from about 8:1 to about 10:1 for immediate relief from chronic arthritis pain.

In yet another embodiment, a method of preparing an arthritis antidote tonic is described. The method includes the steps of providing from about 2 oz. to about 10 oz. of organic apple juice; providing from about 1 gram to about 3 grams of turmeric powder; mixing said apple juice and said turmeric powder; stirring said mixture to form said antidote tonic, wherein the tonic is consumed three times a day for relief from arthritis pain and inflammation.

In yet another embodiment, a method for getting relief from arthritis pain and inflammation is described. The method includes the steps of providing an arthritis antidote composition, the composition including organic apple and turmeric powder, the composition is in the form of one of a tonic, juice, applesauce, capsule, or powder; consuming said composition three times a day for at least one month; reducing intake of said composition upon determining reduced severity of arthritis and maintaining three intakes of said composition on determining high severity of arthritis.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a block diagram view of one potential embodiment of an arthritis antidote organic tonic of the present invention in accordance with the disclosed architecture;

FIG. 2 illustrates a flow chart depicting a process of forming arthritis antidote tonic of the present invention in accordance with the disclosed architecture;

FIG. 3 illustrates a perspective view of arthritis antidote formulated capsules of the present invention in accordance with the disclosed architecture;

FIG. 4 illustrates a perspective view of arthritis antidote applesauce of the present invention for treating arthritis inflammation in accordance with the disclosed architecture;

FIG. 5 illustrates another composition of the arthritis antidote of the present invention in accordance with the disclosed architecture; and

FIG. 6 illustrates a flow diagram depicting an exemplary method of use of various embodiments of the arthritis antidote of the present invention in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for an improved treatment for arthritis that is organic and chemical-free. There is also a long-felt need in the art for an improved treatment for treating arthritis that is safe and cost-effective. Additionally, there is a long-felt need in the art for an arthritis treatment that reduces dependency on conventional arthritis medications and therapies. Moreover, there is a long-felt need in the art for an organic arthritis treatment that is capable of alleviating aches, pains, and stiffness in the joints to promote better mobility. Further, there is a long-felt need in the art for an arthritis treatment that does not require a person to repeatedly visit a medical practitioner. Further, there is a long-felt need in the art for an arthritis treatment that provides immediate relief and helps in improving quality of life of people affected from arthritis. Finally, there is a long-felt need in the art for an arthritis treatment that can be consumed like a tonic and utilizes organic ingredients to improve overall health without the need for chemical medications.

The present invention, in one exemplary embodiment, is a method of preparing an arthritis antidote tonic. The method includes the steps of providing from about 2 oz. to about 10 oz. of organic apple juice; providing from about 1 gram to about 3 grams of turmeric powder; mixing said apple juice and said turmeric powder; stirring said mixture to form said antidote tonic, wherein the tonic is consumed three times a day for relief from arthritis pain and inflammation.

In some embodiments, the disease or condition mediated by organic tonic of the present invention is an inflammatory disease. In various embodiments, the disease is rheumatoid arthritis (RA), reactive arthritis, monoarticular arthritis, osteoarthritis, gouty arthritis, juvenile arthritis, juvenile onset rheumatoid arthritis, juvenile rheumatoid arthritis, or psoriatic arthritis.

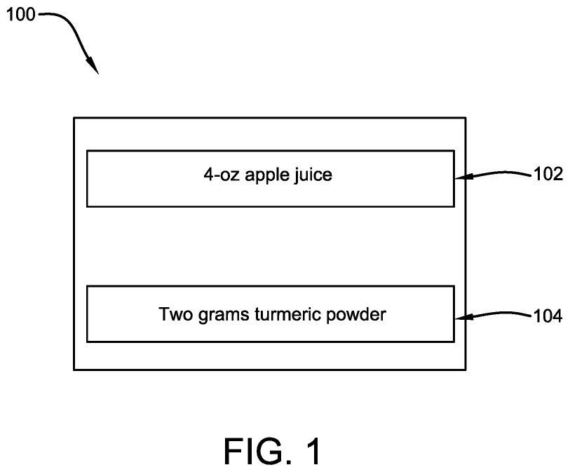

Referring initially to the drawings, FIG. 1 illustrates a block diagram view of one potential embodiment of the arthritis antidote organic tonic of the present invention in accordance with the disclosed architecture. The tonic 100 is configured for self-healing and reduction of pain in the joints caused due to arthritis. The tonic 100 is organic and does not contain any chemicals and is also configured to improve sleep, rest, and breathing to maintain optimal sleep cycles of an individual suffering from arthritis.

More specifically, the tonic 100, in one exemplary composition ratio, includes from about 2 oz. to about 8 oz. of 100% natural apple juice 102 combined with from about 1 gram to about 4 grams of turmeric powder (Curcuma longa) 104 for providing immediate relief from arthritis pain. The tonic 100 is formed by mixing the apple juice 102 and the turmeric powder 104 to formulate the antidote tonic 100. Apple juice 102 has anti-inflammatory properties and helps in reducing inflammation. Turmeric is a natural plant and helps in reducing pain, stiffness, and inflammation related to arthritis.

Turmeric 104 contains a natural chemical compound called curcumin that also has antioxidant properties. More specifically, turmeric 104 when mixed in apple juice 102 neutralizes free radicals which are unstable molecules and occur naturally in human body and suspected to cause painful arthritis inflammation. Turmeric 104 also helps in suppressing human body's response to tumor necrosis factor (TNF), a chemical produced by the immune system that causes inflammation related to rheumatoid arthritis, psoriatic arthritis, and other arthritic conditions.

In another exemplary embodiment, the antidote tonic 100 is commercially available as a combination of 4-oz apple juice bottle and a pouch of 2 grams of turmeric powder. Alternatively, the antidote tonic 100 can be available in formulation in which 2 grams of turmeric powder is pre-mixed in 4-oz apple juice.

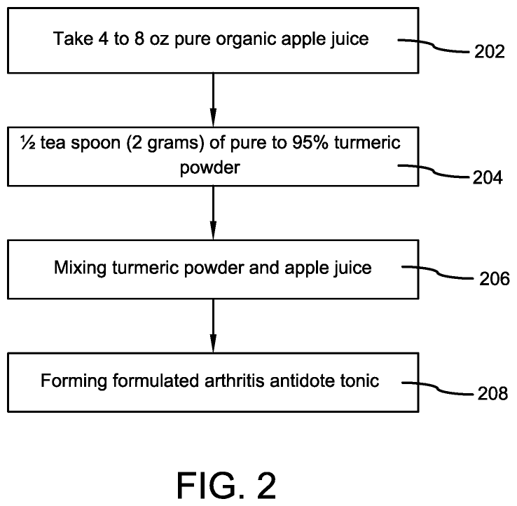

FIG. 2 illustrates a flow chart depicting a process of forming arthritis antidote tonic of the present invention in accordance with the disclosed architecture. For preparing the antidote tonic 100, 100% pure organic apple juice is used which can be extracted directly from natural apples (Step 202). For commercial purposes, the apple juice can be extracted using hydraulic press which includes apple washing, crushing and juice extraction. For personal purposes, the apple juice can be extracted using a mixer or juicer.

In one formulation, 12 teaspoon or about 2 grams of pure to 95% pure turmeric powder is used (Step 204). The turmeric powder can be any organic turmeric powder. The turmeric powder used for commercial production of the antidote tonic 100 is formed by oiling the rhizomes, drying in a hot oven, and then grinding the dried roots. Then, the apple juice and the turmeric powder are mixed, and the mixture is stirred for few seconds (Step 206). Finally, after successful mixing of the apple juice and the turmeric powder, arthritis antidote tonic is formulated for use to provide relief from arthritis pain and inflammation (Step 208).

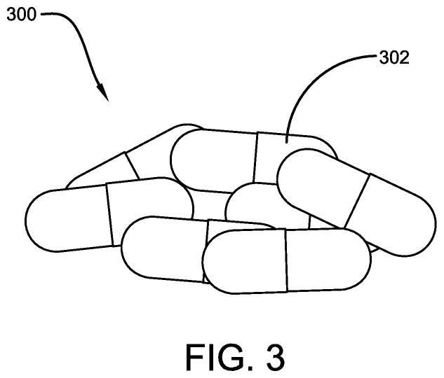

FIG. 3 illustrates a perspective view of arthritis antidote formulated capsules of the present invention in accordance with the disclosed architecture. In the present embodiment, the arthritis antidote is available in the form of formulated capsules 300. Each capsule is water-soluble and contains about 1000 mg turmeric having 90% curcuminoids and organic apple juice extract. Two capsules can be dissolved in water to form a single serving of arthritis antidote for relief from pain and inflammation.

The capsules 300 are marine derived capsules 302 and may have organic certification. For forming capsules, the apple juice extract and turmeric powder are weighed and mixed and encapsulated in capsules using a tablet-making machine. The capsules 300 can be sold in containers containing up to 200 capsules.

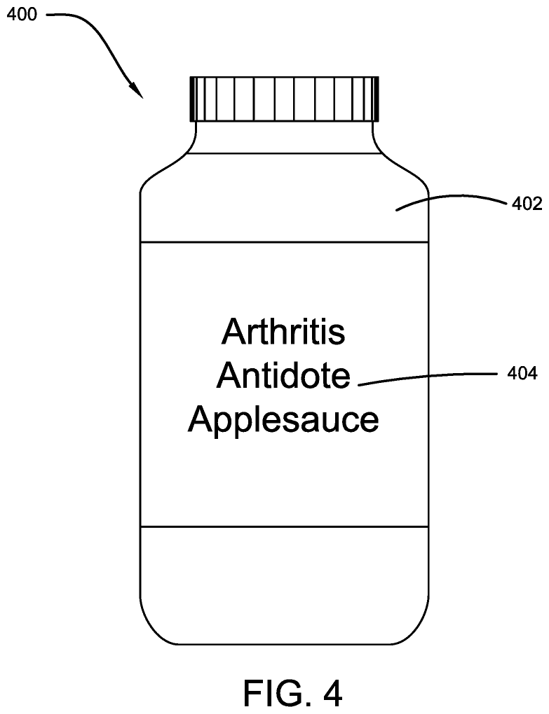

FIG. 4 illustrates a perspective view of arthritis antidote applesauce of the present invention for treating arthritis inflammation in accordance with the disclosed architecture. In the present embodiment, the arthritis antidote is in the form of applesauce 402 which is available in a jar or container 400. The applesauce 402 is made from real and organic apples with added ascorbic acid and turmeric powder. More specifically, the applesauce 402 does not include artificial flavors and is gluten free.

In another exemplary formulation, one tablespoon of the applesauce 402 contains one gram of turmeric powder and 2 tablespoons of the applesauce 402 can be used in one serving for providing immediate relief from arthritis pain. The applesauce 402 may also contain fructose syrup or can be available in unsweetened form. In the present embodiment, the container 400 can have a size from about 15-oz to about 30-oz and can have a logo or any other indicia 404 for marketing and branding purposes.

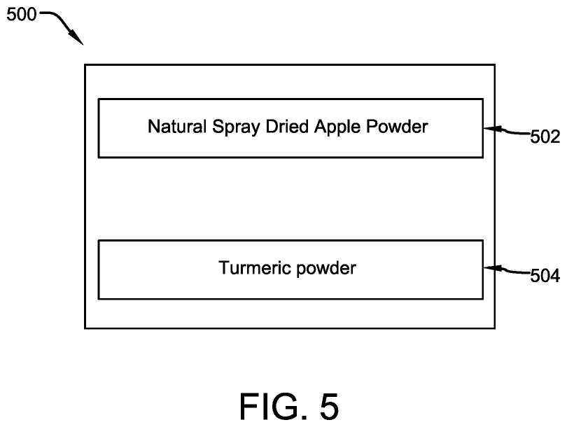

FIG. 5 illustrates another composition of the arthritis antidote of the present invention in accordance with the disclosed architecture. In the present embodiment, the antidote is in powder form. Apple powder can be processed by pressing, drying or dehydrating, and/or grinding the apples into an apple powder. A composition 500 formed of natural dried apple powder 502 and turmeric powder 504 in the ratio in one exemplary formulation from about 8:1 to about 10:1; in another exemplary formulation from about 8.5:1 to about 9.5:1; and, yet in another exemplary formulation from about 8.8:1 to about 9.2:1. The apple powder 502 is 100% pure and natural, vegan, and is GMO and gluten free. More specifically, the apple powder 502 is formed from dehydrated apples and therefore, maintains aroma and taste of natural apples.

The antidote powder 500 can be mixed in water to form arthritis antidote tonic and can be readily used for consumption to get relief from arthritis pain and inflammation. The powder 500 can also be used to make juices, smoothies, jams, pie, and other items. The powder 500 can be packaged in zip lock pouch for maintaining freshness and easy storage.

It should be appreciated that arthritis pain relieving product made from apple juice and turmeric powder can be in the form of juices, applesauce, smoothies, powder, capsules and more. The product is based on natural products and does not use any chemicals and therefore, does not cause any side effects. The product of the present invention helps in alleviating chronic pain due to arthritis, inflammation in joints, dehydration, insomnia, fatigue, and stiffness in joints and muscles.

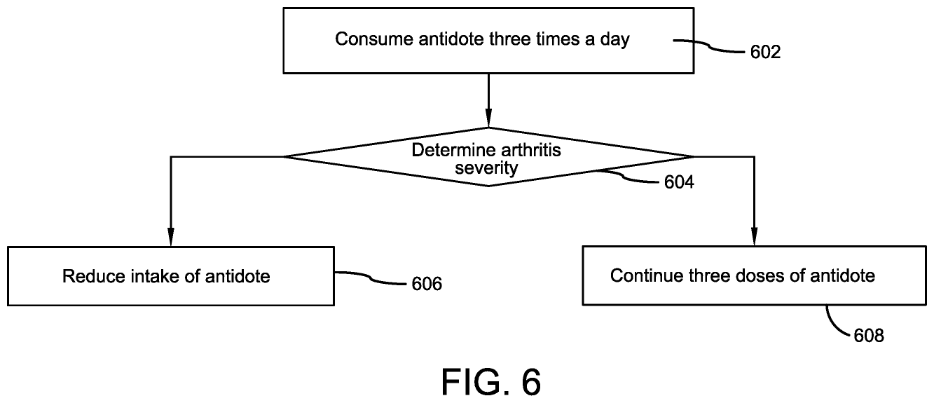

FIG. 6 illustrates a flow diagram depicting an exemplary method of use of various embodiments of the arthritis antidote of the present invention in accordance with the disclosed architecture. Initially, one of the antidote products is selected for consumption and is consumed three times a day for optimal results (Step 602). Preferably, the antidote is consumed after each meal since the antidote can tend to suppress the appetite of an individual. In situations where a person wants to lose weight, then the antidote can be consumed before meals.

After consuming the antidote product for a period of time, a month for example, the user consuming the antidote determines severity of arthritis (Step 604). In situations where the severity is reduced, then, intake of antidote is reduced such as twice on a daily basis (Step 606) else the three doses of antidote are continued to be consumed by the person (Step 608). The antidote in various forms is fast acting and is effective approximately 10 to 15 minutes after consumption for self-healing, reducing of pain in the joints, aiding sleep, and improving breathing.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “arthritis antidote”, “antidote”, “arthritis pain relieving product”, “arthritis antidote applesauce”, “capsule”, and “arthritis antidote powder” are interchangeable and refer to the arthritis antidote composition 100, 300, 400, 500 of the present invention.

Notwithstanding the foregoing, the arthritis antidote composition 100, 300, 400, 500 of the present invention can be of any suitable configuration and natural ingredients as is known in the art without affecting the overall concept of the invention, provided that it accomplishes the above-stated objectives. One of ordinary skill in the art will appreciate the arthritis antidote composition 100, 300, 400, 500 as shown in the FIGS. are for illustrative purposes only, and that many other configurations of the arthritis antidote composition 100, 300, 400, 500 are well within the scope of the present disclosure. Although the arthritis antidote composition 100, 300, 400, 500 important design parameters for user convenience, the arthritis antidote composition 100, 300, 400, 500 may be of any configuration and composition that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A pain reducing tonic configured for self-healing joint pain reduction, the pain reducing tonic comprising:
an organic tonic having a composition ratio from about 2 oz. to about 8 oz. of natural apple juice combined with from about 1 gram to about 4 grams of turmeric powder;
wherein said apple juice packaged in a juice bottle;
wherein said turmeric powder packaged in a pouch;
wherein said turmeric powder is poured from said pouch into said juice bottle; and
further wherein said apple juice is mixed with said turmeric powder to form said organic tonic.

2. The pain reducing tonic of claim 1, wherein said apple juice is extracted from apples.

3. The pain reducing tonic of claim 2, wherein said extraction includes pressing said apples.

4. The pain reducing tonic of claim 1, wherein said organic tonic having a composition ratio including about 4 oz. apple juice mixed with about 2 grams of said turmeric powder.

5. The pain reducing tonic of claim 4, wherein said turmeric powder is at least 95% pure turmeric powder.

6. A method of making and using an organic tonic configured for self-healing joint pain reduction, the method comprising the steps of:
pressing apples to extract natural apple juice;
extracting rhizomes from a turmeric plant;
oiling said rhizomes;
drying said rhizomes;
grinding said rhizomes into a turmeric powder;
forming an organic tonic having a composition in a ratio from about 2 oz. to about 8 oz. of said natural apple juice and about 1 gram to about 4 grams of said turmeric powder; and
mixing said natural apple juice with said turmeric powder to form said organic tonic.

7. The method of claim 6, wherein said organic tonic having a composition in a ratio from about 4 oz. said natural apple juice and about 2 grams of said turmeric powder.

8. The method of claim 7, wherein said turmeric powder is at least 95% pure turmeric powder.

9. The method of claim 6 further comprising a step of drinking said organic tonic.

10. The method of claim 9, wherein said drinking said organic tonic includes a frequency of at least two times per day.

11. The method of claim 6 further comprising a step of adding ascorbic acid and fructose syrup to said organic tonic.

12. The method of claim 11, wherein said natural apple juice is applesauce.

13. A method of making and using an antidote powder configured for self-healing joint pain reduction, the method comprising the steps of:
dehydrating apples;
grinding said apples into an apple powder;
extracting rhizomes from a turmeric plant;
drying said rhizomes;
grinding said rhizomes into a turmeric powder;
forming an antidote powder having a composition in a ratio from about 8:1 to about 10:1 of said apple powder to said turmeric powder; and
mixing said apple powder with said turmeric powder to form said antidote powder.

14. The method of claim 13, wherein antidote powder having a composition in a ratio from about 8.5:1 to about 9.5:1 of said apple powder to said turmeric powder.

15. The method of claim 13, wherein antidote powder having a composition in a ratio from about 8.8:1 to about 9.2:1 of said apple powder to said turmeric powder.

16. The method of claim 14, wherein said turmeric powder is at least 95% pure turmeric powder.

17. The method of claim 13 further comprising a step of mixing said antidote powder in water and drinking a dissolved said antidote powder.

18. The method of claim 17, wherein said drinking said dissolved said antidote powder includes a frequency of at least two times per day.

19. The method of claim 13 further comprising a step of encapsulating said antidote powder in water-soluble capsules.

20. The method of claim 19, further comprising the steps of:
dissolving said water-soluble capsules in water;
mixing said antidote powder in the water; and
drinking a dissolved said antidote powder.
